(19) United States
(12) Patent Application Publication
(10) Pub. No.: US 2014/0147389 A1
(43) Pub. Date: May 29, 2014

(54) Antigen Specific Ultrasound Contrast Medium, a Process for the Preparation Thereof and Its Uses

(71) Applicant: Giuseppe Sabino, Pastorano (CE) (IT)

(72) Inventor: Giuseppe Sabino, Pastorano (CE) (IT)

(21) Appl. No.: 13/812,273

(22) PCT Filed: Jul. 27, 2011
(86) PCT No.: PCT/IB11/53341

§ 371 Date: Jan. 25, 2013

(30) Foreign Application Priority Data
Jul. 28, 2010 (IT) PI2010A000095

(51) Int. Cl. A61K 9/00

(52) U.S. Cl. CPC A61K 9/0009
USPC 424/9.5

(57) ABSTRACT

The present invention refers to an antigen specific echographic contrast medium for diagnostic and/or therapeutic use, a process for the preparation thereof and its uses in ultrasound diagnostic imaging and in therapy. In particular, a contrast medium according to the invention has proven to be useful in selectively carrying drugs and/or other bio-active principles into a diseased tissues and in releasing the same in site, once bond to such tissue and suitably insonated. Moreover, an echographic contrast medium according to the invention allows a differentiation of the diseased tissues from the surrounding (healthy) tissues, by conferring an improved contrastographic enhancement to the diseased tissue alone over an extended period of time.

### TECHNICAL FIELD OF THE INVENTION

The present invention refers to a diagnostic and/or therapeutic antigen specific ultrasound contrast medium, a process for the preparation thereof and its uses in ultrasound diagnostic imaging (commonly referred to as ultrasound diagnostic imaging), as well as in other types of diagnostic imaging and/or in therapy.

In particular, said contrast medium has proven to be useful in selectively carrying and releasing suitable pharmacological substances and/or other bioactive principles, also including non-echographic contrast media, into diseased tissues because of its capability of selectively detecting and disclosing tumoral tissues/bodies, while distinguishing them from the neighbouring (healthy) tissues, by means of conferring an improved contrastographic enhancement to the diseased tissue alone over an extended period of time.

### DESCRIPTION OF THE PRIOR-ART

The ultrasound contrast media (or CM) are substances which can enhance the ultrasound images contrast.

To day, among the most used ones, there are those of the so called second generation, that are substantially constituted by gas microbubbles, preferably of an inert gas (such as, e.g., sulphur hexafluoride, perfluorohexane, perfluorocarbons, air, nitrogen), contained inside a suitable coating; preferably, said coating is under the form of the so called microcapsules/microbubbles/microspheres/microvesicles/microoballoons which are totally or substantially formed by suitable stabilizing substances such as, for example, phospholipids or albumin.

The element that enhances the ultrasonic signal is a gas, which has an echogenicity coefficient well greater than the one of mostly biologic solids and liquids. The most used contrast media are constituted by, for example, gas containing microcapsules/microbubbles/microspheres/microvesicles/microoballoons (hereinafter normally referred to as microspheres, for the sake of simplicity) characterized by a specific resonance frequency f0 (usually measured in MHz), i.e., an oscillation frequency wherein the particle begins to be submitted to cyclic phases of compression and rarefaction. The contrast media of the so called second generation resulted particularly useful because they possess a resonance frequency which is comprised inside the range of the ultrasonic frequencies already commonly used by the existing ultrasound machines.

Once injected into the bloodstream, said media begin to resonate when they are shot by an ultrasound rays beam. Accordingly, they give raise to a reflected ultrasonic beam showing physical characteristics which are caused either by the properties of the injected substance or by the ones of the incident insonating ultrasound beam.

In particular, with reference to the microsphere physical characteristics, if the membrane thereof has a greater thickness and stiffness, it gives raise to a less strong (i.e., less useful) sonic response in comparison to a particle having a thinner and, therefore, more elastic membrane.

On the other hand, a too thin membrane has the disadvantage of being too weak and, then, less resistant to the action of the pressure exerted by the incident ultrasonic beam. In this case, the microspheres could be destroyed (by sonoporation or by cavitation) in a too quick period of time to be able to provide an image which is diagnostically useful.

If the microspheres number that come into contact with the ultrasound beam becomes greater, then a linear increase in the echogenicity coefficient of the individual particle/microsphere is found. That is to say, said coefficient takes a total value which is obtained by multiplying the individual microsphere coefficient by the total number of the microspheres injected into the bloodstream.

As per the incident ultrasound beam, its basic characteristics are frequency and acoustic strength (usually measured in KPa). In particular, acoustic strength is crucial in characterizing the extent of the reflected beam and the type of the particle oscillation.

Precisely, if the contrast medium microsphere is shot by an ultrasound beam having a lower acoustic pressure (on average comprised from 10 to 20 KPa) and a frequency that is equal to the resonance frequency of the contrast medium (CM), it only undergoes shape alterations of a linear type. In this case, the compression phase results, on average, equal to the rarefaction one and the reflected ultrasound signal is spread toward every direction (it is the so called scattering phenomenon). On the contrary, if the incident beam (while still keeping a frequency equal to the resonance one) takes higher values of acoustic pressure (for example, comprised from 40 KPa to 50 KPa), then the rarefaction phase results greater than the compression one. This effect enables the microspheres to generate a reflected ultrasound beam enriched in a series of harmonic frequencies, which are multiple of the resonance one (i.e., 2f0, 3f0, 4f0 and so on). Moreover, an even greater acoustic strength, for example, up to about 1 MPa causes the production of sub-harmonic frequencies (f0/2, f0/5, etc.). Usually, a strength greater than 1 MPa causes the breaking of the microspheres, this effect being used in echographic imaging techniques with the first generation CM.

The reflected ultrasound beam intensity (either measured in Pa or in dB) results directly proportional to the incident ultrasound beam intensity and to the echogenicity coefficient of the medium, and inversely proportional to the radius of the microsphere that constitutes the contrast medium.

The harmonic frequencies, as already mentioned above, are formed by multiples and sub-multiples of the fundamental frequency of the insonating ultrasound beam. Such an harmonics production is substantially caused by elastic alterations that occur in the microsphere shapes. As already shown above, said alterations are generated by compression and rarefaction phases of the microspheres themselves, which are caused by the quality of the acoustic pressure exerted by the ultrasound beam.

In general, the preferred harmonic frequency is the second harmonic one, because it is easily displayable on the ultrasound machines. That is due to the fact that the second harmonic is the nearest frequency to the fundamental one, and then the more properly recognizable.

The echographic probe is able to detect/read this frequency when it is suitably set during the receipt phase. Moreover, depending on the used echographic procedures (thanks to the use of proper softwares), it is possible to analyse the reflected signal that comes from the various structures using a broad range of different technical schemes. For example, it is possible to subtract the harmonics which come from the static tissue, to characterize the signal which only comes from the vascular apparatus, to modulate the signal phase, its width or both of them. The final goal is, in any case, that of selectively recognizing and differentiating, together with a considerable contrast enhancement, only the anatomical structures which are soaked with the contrast medium, in comparison to the ones that are not.

From a technical point of view, when the CM is injected into the blood stream and the echographic probe is placed upon the specific body tissue area that has to be analysed, it is possible to follow the microspheres movement in three phases:
arterial phase (from 10 to 35 seconds);
parenchymal phase (from 40 to 120 seconds);
venous phase (from 120 seconds on, until the disappearance of the contrast medium from the circulation of the organ under examination).

In addition to the capability of creating a valid imaging contrast, it is well known that the echographic contrast media may also contain pharmacological substances and/or other bio-active principles, including other types of non-echographic contrast agents, and that they can release them into a diseased tissue, in which they are gathered, once properly insonated with a suitable ultrasound beam.

Hence, it would be very useful to have an echogenic contrast medium sufficiently stable and site-specific, for example able to importantly and selectively distinguish with time a diseased tissue (e.g., tumoral) from a neighbouring healthy tissue and able to release a pharmacological substance into said tissue in a controlled and most of all modulated way. In other words, on the one hand, it would be very useful to have an echogenic contrast medium stable and able to selectively give a high contrastographic enhancement to the diseased tissue, in comparison to the neighbouring healthy tissue, at least for all the necessary time for successfully carrying out the desired echographic diagnostic examination. On the other hand, it would equally be very useful whether said echogenic contrast medium were also able to selectively release a suitable drug into said diseased tissue in a manner that is capable of being modulated by the operator in view of remedying the complications that can derive from a wrong infusion rate of the drug at issue.

Attempts of giving an answer to the above mentioned need have been made.

Thus, by way of an example, microspheres, containing an inert gas, have been made in which one or more receptors, specific for one or more antigens present in the diseased/tumoral tissue, are contained.

In this type of contrast media of the known art, said receptors (such as, for example, monoclonal and non-monoclonal antibodies and/or their fragments, e.g. Fab's, i.e. Fragments Antigen Binding) are incorporated into the microsphere membrane, for example, inserted among the phospholipid chains components thereof. In such a way, attempts were made to favour/promote the formation of a specific and possibly strong bond between the receptor and the corresponding antigen, which is present inside the tissue to be submitted to imaging, when the contrast medium perfuse it. Such a procedure may potentially result useful also for carrying suitable drugs, incorporated inside the microsphere, which will successively be released into the desired tissue(s) once this/these are submitted to suitable insonation at the moment in which they are perfused by the bloodstream containing the echographic CM.

To day, such a system is still used only at the experimental stage and it has not been possible to conveniently reproduce it in the human organism yet.

One of the main drawbacks of the above described system consists in the fact that the drugs which are contained inside the microbubble can not be released in a modulated way, fit to the patient needs, but are released all at once through only one interaction between the microspheres and the incident ultrasonic beam; in such a way the safety of the administration is seriously endangered in those clinical situations in which a slow rate of infusion or administration of the drug is necessary, for example, during the thrombo-lytic therapy of the carotid plaques.

For example, WO 2008/110958 discloses a composition comprising ultrasound sensitive particles which comprise a sub-group of particles made of liposomes for the selective release of biologic material. However, it does not disclose, nor even suggests, the possibility of using concentric microbubbles which membranes are substantially structurally formed by a receptor which is specific for a certain antigen.

WO 2007/008220 discloses a method of liposomal bonding both inside and outside the basic microbubble, which is neutral or albumin coated, but it does not describe the use of more than one than one interfaces for increasing the efficacy of sonoporation nor does it cites, nor even does it suggests, the possibility of using concentric microbubbles which membranes are substantially or mainly structurally formed by a receptor which is specific for a certain antigen.

Liang H-D et al.: “Sonoporation, drug delivery, and gene therapy”, Proceedings of the Institution of Mechanical Engineers. Journal of Engineering in Medicine. Part H, Mechanical Engineering Publications, Ltd., London GB, Vol. 224 no. 2, (Jan. 1, 2010), pages 434-361, scrupulously describe the most recent innovations in the ultrasonic field; however, they do not mention, nor even suggest, the possibility of using concentric microbubbles in which the membranes are substantially or mainly structurally formed by a receptor which is specific for a certain antigen.

### Technical Problem

Therefore, to day it is still strong and deep-felt in the medical field the need for an echographic contrast medium which, following a proper insonation type, is able to release, in a modulated and controlled way, a pharmacological or bio-active substance associated to the same, after selectively and properly, i.e., strongly enough and, consequently, for a sufficiently long/useful period of time, forming a bond with the only tissue to be submitted to imaging (usually, the diseased one) in comparison to the neighbouring tissues (usually, the healthy ones), so as to selectively confer to the same a substantially high and prolonged contrastographic enhancement, at least for all the necessary and sufficient time for successfully carrying out the desired, selective, echographic diagnostic imaging and for simultaneously realizing the desired selective therapeutic treatment and modulated according to the need.

### SUMMARY OF THE INVENTION

The Applicant has now completely unexpectedly found that, by preparing an echographic contrast agent consisting of a number of suitable different microspheres, which are inserted one into the other, substantially concentric with respect to each other, and in which said microspheres are each one coated by a membrane comprising as a constitutive element thereof a suitable amount of a suitable receptor which is antigen-specific for a certain antigen and contain a suitable inert gas and a suitable bio-active principle, it is possible to give an adequate answer to the technical problem above described.

Therefore, it is one embodiment of the present invention an antigen-specific echographic contrast agent consisting of at least two microspheres different with each other, and being the one inserted inside the other, in which each of said microspheres is coated by a membrane formed by suitable amounts of a high affinity receptor specific for a certain antigen, which is present inside the tissue to be submitted to imaging, and of a stabilizer thereof, and contains a suitable inert gas, as reported in the appended independent claim.

Then, it is one other embodiment of the present invention an echo-contrastographic pharmaceutical composition comprising the above contrast medium, as reported in the appended independent claim.

Moreover, it is one other embodiment of the present invention a process for preparing the above contrast medium, as reported in the appended independent claims.

It is a further embodiment of the present invention the use of the above contrast medium for carrying out the selective echographic imaging of a tissue (preferably, a diseased/tumoral tissue) in comparison to the surrounding (preferably, the healthy ones) tissues, as reported in the appended independent claim.

Yet, it is a further embodiment of the present invention the use of the above echographic contrast medium as a selective carrier of drugs and/or other bio-active principles, as reported in the appended independent claim.

Moreover, it is a further embodiment of the present invention the use of the above echographic contrast medium for releasing a pharmacologically active principle to a diseased tissue in a selective and modulated way, as reported in the appended independent claim.

Other embodiments of the present invention are described in the appended dependent claims.

### DETAILED DESCRIPTION OF THE INVENTION

The antigen-specific echographic contrast agent according to the present invention consists of at least two (or even more) microspheres, different among each other and placed each one inside the other one, in which each one of said microspheres:
is coated by a membrane, different for every microsphere, and substantially constituted by an effective amount of at least one high affinity receptor, which is specific for a certain antigen present in the tissue to be submitted to imaging and/or to pharmacological treatment, and by an effective minimal amount of at least one stabilizer, being the reciprocal weight ratio receptor:stabilizer comprised from 10:1 to 1:1; and
contains an inert gas, which is the same or different for each microsphere.

Preferably, the contrast agent according to the invention consists of two microspheres, which are different between each other, are placed the one inside the other one and are substantially concentric between each other.

The membranes which are coating each one of said at least two microspheres are different among them: precisely, the membrane coating the outer microsphere is different from the membrane coating the inner microsphere.

Said membranes contain an effective amount of at least one antigen-specific receptor which has high affinity for a certain antigen that is present in the tissue to be submitted to imaging and/or pharmacological treatment. More preferably, said membranes contain only one single antigen-specific receptor. As a matter of fact, both of the microspheres are antigen-specific.

In a particularly preferred embodiment of the invention, said receptor is the same for every type of membranes, i.e., both for the membrane coating the outer microsphere and for the membrane coating the inner microsphere.

For example, said receptor may be selected from the group comprising: antibodies, either monoclonals or non-monoclonals, and/or their fragments, such as Fab's, and/or VEGFR (Vascular Endothelial Growth Factor Receptor) receptors specific for endothelial cells, myocardial cells, proper lamina cells, interstitial cells, cells which are expressed in the atheroschlerotic plaques, as well as specific receptors for immunoglobulines, complement fragments, peptide and lipid hormones, neurotransmitters.

Particularly preferred receptors are the antibody Fab fragments having high affinity, which are specific for a certain antigen. The preference given to the use of antibody Fab fragments, in comparison to a whole antibody, is due to the fact the antibody, deprived of its Fc fragment (crystallizable fragment), loses its cytotoxic properties and activating the complement molecules. In this way, it has been possible to remove, or at least substantially reduce, the onset of side effects.

Preferably, said membrane which is coating each one of the said microspheres further contains a minimum effective amount of at least one substance that has a stabilizing action (in some of the realizations of the inventions it is in any case possible to also have a mixture of two or more stabilizers). Said at least one stabilizer substantially exerts its action on the membrane, but also on said antigen-specific receptor, thus allowing the membrane (i.e., precisely the assembly receptor-stabilizer) to keep intact in the organism its substantially spherical structure for an appropriate period of time, so that it can make its way towards the target site and effectively bind to it for all the time necessary for carrying out the diagnostic and/or therapeutic procedure.

Said at least one stabilizer is different for each type of membrane: precisely, the stabilizer contained in the membrane coating the outer microsphere is different from the stabilizer contained in the membrane coating the inner microsphere(s).

Said stabilizer is, for example, selected from the group comprising: albumin, phospholipids, galactose, palmitic acid, cianoacrylate. Preferred stabilizers resulted to be albumin, phospholipids, palmitic acid, galactose and the mixtures thereof; particularly preferred resulted to be albumin, phospholipids and a mixture palmitic acid/galactose.

Albumin resulted even more preferred because it exerts a strong stabilizing action on the proteins (antibodies, Fab's and so on) and, then, may efficiently amalgamate, even at low doses, the protein molecules that form the membranes of the microspheres, without excessively diminishing the concentration and, thus, the efficiency thereof.

The amount of the added stabilizer is the lowest necessary: in any case, just the amount that is sufficient to stabilize the membrane of the microsphere in the proper/desired way. Usually, a reciprocal ponderal ratio receptor:stabilizer comprised from 10:1 to 1:1 is appropriate; preferably, said ratio is comprised from 7.5:1 to 1.5:1; more preferably, from 5:1 to 2:1; even more preferably, from 4:1 to 2.5:1. In a particularly preferred realization of the invention, said reciprocal ponderal ratio receptor:stabilizer is of 3.5:1; preferably, it is of about 3:1.

Practically, the membrane of the microsphere may contain a maximum receptor amount up to about 91% by weight, with respect to the total weight of the membrane. Preferably, depending on the desired realization, the amount of said receptor may reach about 88% or 83% or 80% or 77% by weight, with respect to the total weight of the membrane; more preferably 75% by weight, with respect to the total weight of the membrane. On consequence, from the above description it clearly results that said antigen-specific receptor, contrary to what is known and practiced in the art, is not inserted, on average in a limited amount, in the membrane or upon the external surface thereof or inside the same, but is a structural integral part thereof, while being the amount of the stabilizer the lowest possible (depending on the type of the antigen-specific receptor) necessary to give the desired stability to the membrane itself.

Indeed, said very low amount of stabilizer has unexpectedly shown to be more than sufficient to give a significant stability to the membrane of the microsphere. Thus, in a completely unexpected way in comparison to what known in the art, it has been possible to obtain very stable microspheres in which the membranes are mainly or substantially constituted by an antigen-specific receptor (preferably a Fab of an antibody). As a consequence, said microspheres resulted to be endowed with a specificity toward the desired target site, tissue or organ which is substantially higher (at least higher than 10-15%, but, possibly, also ≧20% and even more), in comparison with the antigen-specific receptor-containing microbubbles known in the art. It is then a particularly innovative and advantageous aspect of the present invention, the realization of microspheres, which resulted unexpectedly stable in spite of the fact that their membranes are structurally formed by a small amount of a stabilizer and by a high amount of an antigen-specific receptor which guarantees a substantially better specificity in comparison to the microspheres of the known art.

Just by way of an example, in a preferred realization of the invention, albumin is the stabilizer contained in the membrane that is coating the outer microsphere, while the phospholipids are the stabilizer contained in the membrane that is coating the inner microsphere.

In another preferred realization of the invention, the situation is the opposite: i.e., albumin is the stabilizer contained in the membrane that is coating the inner microsphere, while the phospholipids are the stabilizer contained in the membrane that is coating the outer microsphere.

In other preferred realizations the membrane that is coating one of the microspheres may, for example, contain a mixture palmitic acid/galactose as a stabilizer. In said mixture the two components are for example present in a reciprocal ponderal ratio (w:w) comprised from 10:1 to 1:10; preferably, comprised from 5:1 to 1:5; more preferably of about 1:1.

The gas contained into each of said at least two substantially concentric microspheres, which form the antigen-specific echographic contrast agent according to the present invention, is the same or different for every type of microsphere; precisely, the gas contained into the outer microsphere is the same or different from the gas contained into the inner microsphere(s).

Said gas is, for example, selected from the group comprising: sulphur hexafluoride, perfluorohexane, perfluorocarbons, air, nitrogen. Particularly preferred resulted to be sulphur hexafluoride, air and perfluorohexane; more preferably, sulphur hexafluoride and air.

Just by way of an example, in a preferred realization of the invention, sulphur hexafluoride is the gas contained into the outer microsphere, while air is the gas contained into the inner microsphere.

In another preferred realization of the invention, the situation is the opposite: i.e., sulphur hexafluoride is the gas contained into the inner microsphere, while air is the gas contained into the outer microsphere.

In a further preferred realization of the invention, sulphur hexafluoride or air or perfluorohexane are contained either into the outer microsphere or into the inner microsphere.

In a particularly preferred realization of the invention, said antigen-specific echographic contrast agent according to the present invention further comprises an effective amount of at least a bio-active substance, for example selected from: antigen-specific receptors different from the main one; drugs (just by way of an explanatory, but absolutely non-limiting, example, antitumoral ones); other types of substances, such as, molecules used in molecular imaging, radiologic contrast media for conventional radiology, computerized tomography, magnetic resonance, nuclear medicine; biologically active molecules such as hormones, vitamines; genetic material, such as nucleosides, nucleotides; synthetic substances of a peptidic, lipidic, glucidic nature, so that the resulting echografic contrast medium (CM) may be simultaneously also used as a selective transmitter or carrier for said substances. In particular, the above mentioned molecules used in diagnostic imaging are preferably selected from: magnetite nanoparticles, iodinated compounds, complex paramagnetic ions.

Said at least one bio-active substance is inserted into or associated/bonded, preferably according to known technologies in the field, to each of said at least two microspheres of which the contrast agent according to the present invention consists. Preferably, said bio-active substances are inserted in the membrane that is coating each microsphere and/or inside the same.

Just by way of a preferred, but absolutely non-limiting example of the invention, it is an embodiment of the invention an echographic contrast agent consisting of: one outer microsphere constituted by a bubble of gas, such as air or sulphur hexafluoride or perfluorohexane, coated by a membrane of Fab's from antibodies which are stabilized with an effective minimum amount of albumin, for example in a preferential ponderal ratio of 3:1; one inner microsphere inside the preceding one (substantially concentric with it) constituted by a bubble of gas, such as air or sulphur hexafluoride or perfluorohexane, coated by a membrane of Fab's from antibodies which are stabilized with an effective minimum amount of phospholipids, for example in a preferential ponderal ratio of 3:1.

Said two concentric microspheres may further contain an effective amount of bio-active agents such as drugs, contrast media, biological molecules and so on as above described.

Depending on the need, it is also possible to prepare the reverse formulation.

For example, in another embodiment of the invention, it is possible to realize an echographic contrast agent by reversing the chemical components of the structure. Thus, said agent will consist of:
one outer microsphere constituted by an antigen-specific substance (preferably a Fab) stabilized by phospholipids in a ponderal ratio preferably of 3:1 and containing sulphur hexafluoride as a gas and, in case, suitable amounts of bio-active agents and/or pharmacological substances as above described;
one inner microsphere, placed inside the preceding one, constituted by an antigen-specific substance (preferably a Fab) stabilized by albumin in a ponderal ratio preferably of 3:1 and containing sulphur hexafluoride as a gas and, in case, suitable amounts of bio-active agents and/or pharmacological substances as above described.

The preferred amounts of said bio-active agents and/or pharmacological substances will change according to the type of cell/tissue/organ and/or disease to be treated and in any case already form part of the therapeutic knowledge of the skilled clinical of the field.

Always by way of further examples, non-limitative of the invention, the echographic contrast agent of the invention may consist of two concentric microspheres in which:
a)
the outer microsphere contains air as an inert gas and a mixture of palmitic acid/galactose, in the above mentioned reciprocal ponderal ratio, as a membrane stabilizer, and
the inner microsphere contains sulphur hexafluoride as an inert gas and phospholipids as membrane stabilizers;
b)
the outer microsphere contains air as an inert gas and albumin as a membrane stabilizer, and
the inner microsphere contains air as an inert gas and a mixture of palmitic acid/galactose, in the above mentioned reciprocal ponderal ratio, as a membrane stabilizer;
c)
the outer microsphere contains perfluorohexane as an inert gas and albumin as a membrane stabilizer, and
the inner microsphere contains sulphur hexafluoride as an inert gas and a mixture of palmitic acid/galactose, in the above mentioned reciprocal ponderal ratio, as a membrane stabilizer;
d)
the outer microsphere contains air as an inert gas and albumin as a membrane stabilizer, and
the inner microsphere contains sulphur hexafluoride as an inert gas and a mixture of palmitic acid/galactose, in the above mentioned reciprocal ponderal ratio, as a membrane stabilizer;
e)
the outer microsphere contains sulphur hexafluoride as an inert gas and a mixture of palmitic acid/galactose, in the above mentioned reciprocal ponderal ratio, as a membrane stabilizer, and
the inner microsphere contains perfluorohexane as an inert gas and phospholipids as membrane stabilizers;
f)
the outer microsphere contains sulphur hexafluoride as an inert gas and a mixture of palmitic acid/galactose, in the above mentioned reciprocal ponderal ratio, as a membrane stabilizer, and
the inner microsphere contains air as an inert gas and phospholipids as membrane stabilizers;
g)
each one of the above contrast agents from a) to f) in which the composition of the outer microsphere and the one of the inner microsphere have been reversed.

Preferably, in said contrast agents from a) to g) the ponderal ratio between the total amount of the substance(s) stabilizing the membranes and the Fab is about 1:3, thus confirming the completely unexpected use of a considerably high Fab amount, in view of the present knowledge in the field.

More preferably, said contrast agents from a) to g) further contain an effective amount of bio-active agents such as drugs, contrast media, biological molecules and so on by adopting the same way and amounts above described.

From the dimensional point of view, one individual microsphere taken as such has an average diameter comprised from ≦1 μm to <7 μm, in all its possible realizations; more preferably, said average diameter is comprised from ≦2 μm to <4 μm; even more preferably it is ≦3.5 μm.

In its turn, the membrane thickness of the outer microsphere does not exceeds on average 400 nm; preferably, it is <300 nm: more preferably, it is <200 nm.

Also the membrane thickness of the inner microsphere does not exceeds 400 nm; preferably, it is <300 nm: more preferably, it is <200 nm.

On consequence, the total average diameter of the contrast agent of the present invention (i.e., the substantially spherical ensemble of said at least two concentric microspheres which the echographic contrast agent of the present invention consists of) does not exceed 8 μm; preferably, it is <7 μm; more preferably it is <6 μm.

Said value does not exceed the red blood cells average diameter (of about 8 μm) and, therefore, it guarantees the safety of the contrast medium of the invention.

Moreover, being lower than 10 μm, it also guarantees that the particles of said contrast medium are able to cross the lung barrier.

As a consequence of what has been above disclosed, it is therefore one embodiment of the present invention also an echographic contrastographic composition comprising a contrast agent in accordance with all what has been above described.

Said contrast medium is preferably prepared in accordance with the known formulative procedures of the field.

In one embodiment of the invention, the components, or the precursors, of the contrast medium according to the invention may suitably be individually prepared and pre-packed into a suitable kit (and distributed to the physician in such a form), so that they can be easily put together and reconstituted just before the use, for example, by suitably mixing and gently stirring said components/precursors. Said kit is prepared and structured, mutatis mutandis, similarly to what is usually made with the already known and marketed diagnostic kits.

The process for preparing the microspheres and the contrast agent according to the present invention is preferably carried out by adopting known preparative technologies, which are commonly used for preparing the gas containing microbubbles of the echographic contrast media already known in the art.

Thus, in a preferred embodiment of the invention, the inner microsphere membrane is made by means of a traditional process comprising at least one phase in which a powder of a lyophilized substance constituted by an effective amount of at least one antigen-specific receptor, mixed with a suitable amount of a stabilizing substance selected from the group comprising: phospholipids, albumin, galactose, palmitic acid, cianoacrylate and/or mixtures thereof (preferably, phospholipids, albumin, galactose, palmitic acid), is properly stirred, for example, with a physiological solution under an atmosphere of an inert gas (preferably, sulphur hexafluoride, air, perfluorohexane) for the period of time sufficient to obtain microspheres of said gas coated by an antibody/antigen-specific membrane which contains said antigen-specific receptor (preferably a Fab from an antibody).

Such a membrane may reproduce the structure of some of the CM known in the art, hence the same is synthesized by using the new methodologies, which are commonly used for their preparation.

In its turn the realization of the outer microsphere membrane comprises at least one phase in which a lyophilized powder of a mixture Fab/stabilizer, preferably in a reciprocal ponderal ratio of 3:1, is mixed, under an atmosphere on an inert gas (preferably, sulphur hexafluoride, air, perfluorohexane), for example, in a sealed vial, with a physiological solution (0.9% of NaCl) and in the presence of the (innerer) microspheres previously obtained, and properly stirred for a few minutes at room temperature, for example, manually or under sonication, to give the substantially spherical nucleous of the above described contrast agent of the invention.

Just by way of an example, absolutely non-limiting of the invention, said process preferably comprises a first phase a) in which:
a) a lyophilized powder formed by a mixture constituted by at least one antigen-specific receptor (a Fab) and by an amount of phospholipids (about 10-20 mg) in a reciprocal ponderal ratio of 3:1, is mixed, under a sulphur hexafluoride atmosphere, for example, in a sealed vial, with about 2.5 mL of a physiological solution (0.9% of NaCl) and properly stirred for a few minutes, for example manually or under sonication, at room temperature, to give a first microsuspension of said preformed echogenic microspheres (the so called inner microspheres), having a single phospholipid membrane, and containing sulphur hexafluoride.

Said first phase a) is followed by a second phase b) in which:
b) to the microspheres deriving from the preceding phase a) a lyophilized mixture is added which is constituted by Fab/albumin 3:1 w:w (about 10-20 mg), under an atmosphere of sulphur hexafluoride or of another inert gas, in a sealed vial with about 2.5 mL of a physiological solution (0.9% of NaCl). The whole is properly stirred for a few minutes, for example manually or under sonication, at room temperature, to give a second micro-suspension containing the concentric microspheres (consisting of one inner microsphere and of one outer microsphere, different from the preceding one) object of the present invention.

The maximum storage time (in a sealed vial) of the microbubbles of the art is of about 6 hours, once they have been prepared.

In its turn, their permanence time in the peripheral haematic bloodstream is of about 6 minutes.

In comparison to what is known, the stability of the concentric microspheres of the echographic contrast medium according to the present invention on average resulted considerably higher (preferably, from 1.1 to 5 times higher) and, thus, better, than the one of the microbubbles of the art. On average, the permanence time of the concentric microspheres of the echographic contrast medium according to the present invention in the peripheral haematic bloodstream is assessable to at least 7 minutes; preferably, to at least 8 minutes; more preferably, to at least 9 minutes; even more preferably, to 10 or more minutes.

In view of the above unexpected characteristics shown by the echographic contrast agent of the present invention it has unexpectedly been possible to show that the structure thereof has made it possible to obtain either its particularly prolonged specific permanence inside the tissues that express a certain antigen for which the receptors (Fab's) present in the membranes of the concentric microspheres of the agent of the invention are specific, or the release of a drug contained inside the two microspheres in a targeted and controlled way.

Actually, the membranes of the at least two concentric microspheres have a substantial difference in their chemical structures in term of constituents and that resulted in a differentiation of their visibility at the echographic examination and in an effective modulation of the drug release. This happens because it is necessary to apply different characteristics of the ultrasonic beam depending on whether it is desired to produce the cavitation of one or of the other of the membranes.

In other words, in order to produce, for example, the cavitation of a microsphere made of air coated with albumin, an acoustic power is required which is different from the one that is needed for obtaining the same effect on a microbubble formed by sulphur hexafluoride coated by phospholipids. Thus, after provoking (by applying a suitable acoustic power) the cavitation of the outer microsphere membrane, followed by the corresponding release of the drug contained in it or associated to it, it is possible to successively produce (by applying a suitable different acoustic power(s)) also the cavitation of the inner microsphere(s) membrane(s) together with the corresponding further release of the drug contained in it/them or associated to it/them. In this way it has been possible to obtain a well more prolonged drug release (and, therefore, a prolonged and gradual absorption of the same by the target tissue) in comparison to what would have happened in case the release were provoked only by a single homogeneous membrane.

Thus, the inventive rational of the use of the at least two concentric microspheres of the invention, formed by membranes having a different chemical composition and by the same or different gases, lies in the fact that the membrane chemical constituent and the gas contained in it affect the characteristics of the acoustic beam used for provoking the cavitation in a different way.

Thus, it has been necessary to use acoustic beams having a different potency depending on the type of the microspheres (i.e., depending on the type of membrane and of gas contained in it) that it is desired to submit to cavitation. In such a way it has been possible to cause the sequential cavitation of the different membranes of the at least two concentric microspheres of the invention by changing the acoustic power of the insonating beam, so as to obtain a longer and modulated pharmacological release.

On average, the acoustic power of the insonating beam is ranging from 0.1 to 1 MPa; preferably, from 0.1 to 0.9 MPa; more preferably, from 0.2 to 0.83 MPa, depending on the type of the chemical component of the membrane (and of the type of gas) to be submitted to cavitation.

In its turn, the ultrasonic beam frequency is on average ranging from 1 to 3 MHz; preferably, from 1.1 to 2.9 MHz; more preferably, from 1.15 to 2.85 MHz.

Just by way of an example, absolutely non-binding or non-limiting of the applicative potential of the invention, an echo-contrastographic agent of the invention consisting of two concentric microspheres, which are formed, the outer one, by Fab and phospholipids (in a ponderal ratio 3:1 and containing sulphur hexafluoride and one antitumoral as an active principle), and the inner one, by Fab and albumin (in a ponderal ratio 3:1 and containing air and one antitumoral as an active principle), was submitted to treatment with ultrasounds.

Said agent was firstly insonated with an ultrasonic beam of a frequency comprised from 1.10 to 2.85 MHz and an acoustic power comprised between 0.2 and 0.4 MPa (it caused the poration and the subsequent cavitation of the outer microsphere phospholipid membrane, together with the corresponding release of the drug contained in it or associated to it), and then with an ultrasonic beam of the same frequency and having an acoustic power of 0.83 MPa (it caused the poration and the subsequent cavitation of the inner microsphere albumin membrane, together with the corresponding release of the drug contained in it or associated to it).

In such a way, it was shown that it was possible to cause the sequential cavitation of the two different membranes by suitably changing the acoustic power of the insonating beam, and thus obtaining a prolonged and modulated pharmacological release, into the region in which the contrast agent gave rise to an extremely selective and strong bond.

The contrast agent formed by the at least two concentric microspheres in which both/all of them are specific for a certain antigen, according to the present invention, resulted able to remain fastened to the antigen-expressing tissue for a time considerably more longer than the traditional contrast medium particles.

As a consequence of it, the structure of said tissue resulted more positive to the contrastographic enhancement, and for a more prolonged period of time, in comparison to the other surrounding tissues. On average, this selective and prolonged contrastographic enhancement is detected starting from 120 seconds from the contrast medium injection. In its turn, in a completely unexpected way, the selective increase of the enhancement occurs also before 120 seconds from the administration.

All this can be ascribed to the fact that, through the innovative structure of the contrast agent of the invention (mainly formed by high affinity antibodies, specific for a certain antigen, as above described), it has been possible to confer a superior bonding strength to these two immunological elements (antibody:antigen), as well as a superior stability to all of the microspheres.

Then, a particularly important aspect of the present invention lies in the fact of being able to carry the pharmacological substances contained inside (or associated to) the internal cavities of the at least two concentric microsphere in a controlled and modulated way.

This is achieved thanks to a differentiated insonation which is carried out with two applications of ultrasonic beams having different acoustic powers, and being able to submit to sonoporation and cavitation the concentric microspheres membranes in a modulated way (i.e., in a sequence). Precisely, for example, in order to produce the cavitation of an air microsphere coated by albumin, a different acoustic power is required in comparison to the one necessary for obtaining the same effect on a microsphere formed by sulphur hexafluoride coated by phospholipids. Thus, after provoking the cavitation of the outer microsphere membrane, it has been possible to sequentially provoke the cavitation of the inner microsphere membrane so as to obtain a drug more prolonged release (and then a prolonged and gradual absorption of the same by the tissues), in comparison to what would have occurred in the case in which the release would have happened only from a single homogeneous layer.

It is then an object of the present invention also a method for specifically/selectively releasing in a prolonged and gradual way a drug to a site, a tissue or an organ by administering a contrast agent according to the invention to the organism and then submitting it to a specific and differentiated with time cavitation, through the application of ultrasonic beams which are different in potency and/or intensity.

Preferably, the contrast agent of the invention is formulated as an injectable suspension in a suitable physiologically acceptable aqueous medium to give the desired echo-contrastographic medium.

The contrast medium according to the present invention allows many various therapeutic applications. For example, the thrombolytic therapy in patients suffering from ischemic ictus may show a lot of complications, which are related to the pharmacodynamics of the used substances (that cause the well known haemorrhagic side effects) but also to the speed with which the thrombus formation is lysed. Thus, a therapeutic method able to modulate the speed of the transfer of the drug from the blood stream to the lesion may result useful in the clinical practice. In the treatment of the ischemic ictus, the contrast medium having at least two concentric microspheres

In the treatment of ischemic ictus, the contrast medium having at least two concentric microspheres which are susceptible of a differential application of ultrasonic beams in terms of acoustic power represents a protection suitable both for reducing the dangers related to the infusion rate, and for fulfilling the need to use a local-regional approach (which resulted very effective from the studies concerning the ischemic ictus thrombolysis carried out by intra vascular arterial way).

The same line of reasoning is valid for the thrombolytic therapy during the limb peripheral ischemia or the myocardial ischemia.

Moreover, various diagnostic applications for such a CM exist in particular in the characterization and treatment of neoplasias that regard organs which are easily through ultrasound examination, such as, for example, testicle, liver, thyroid, breast, the limb scheletal muscles. Through this echographic contrast medium, it is possible to characterize the lesions both by aiming at neoplastic markers and aiming at molecules which are iper-expressed at the endothelial level during the neo-vascularization process.

Said contrast medium may also be used as a carrier of drugs which have specific receptors for molecules, expressed at the endothelial level, representing neoplastic neo-vascularization markers.

Similarly, said contrast medium may be proposed as a specific contrast medium for tumoral markers, being well known the local micro-metastasization phenomenon, which provokes a neoplastic cells diffusion through the vascular way into the surroundings close by the tumor.

Absolutely non limiting examples of possible, advantageous clinical applications of an echographic contrast medium according to the present invention are briefly described in the following section, in order to explain the broad applicative potential thereof.
The testicle embryonic carcinoma cells show positiveness to antigens such as α-fetus-protein (AFP) and chorionic gonadotropin (HCG). The selective study of a testicular mass positiveness to these two antigens, suitably charged upon the anticorpal external capsule of a CM according to the present invention, will render it possible to better diagnose, or to exclude from the diagnostic hypothesis, a mixed type germ cells tumor (such as exactly the embryonic carcinoma), without being compelled to resort to surgical probing, which will result of no use and dangerous to the patient.
Many epithelial tumours express cytokeratins: tumours such as the mammary ones could be typified for cytokeratin 8, 18 or 19. Moreover, cytokeratin 5 or cytokeratin 6 could be detected in the case of a squamous cell tumor.
The study with an α-fetus-protein specific echographic CM according to the invention could offer advantages in the diagnostic process of the hepatic neoplasias.
Ca¹²⁵, charged on the echographic CM according to the invention, could be used for studying masses which are suspicious for malignant tumor of uterus or ovaries.
CEA (Carcino Embryonic Antigen) could be used for confirming or excluding the presence of metastasis from colo-rectal carcinoma at an hepatic level.
The study of certain CD (Differentiation Class) antigens could help with the diagnosis of blood cells tumours.
The molecules expressed during the neo-vascolarization process are fundamental and characteristics in the malignant neoplasias growth process. Anti-VGEF antibodies would advantageously help with the identification of this phenomenon.

In particular, the use of these echographic contrast media looks as particularly promising in the pediatric age, in which the utilization of less invasive diagnostic techniques is without doubt more requested and safer. Actually, the higher biological specificity/selectivity of the microbubbles of the present invention looks as particularly promising for limiting the use of biopsy; and this is surely desirable for the pediatric age.

The invention claimed is:

1. An antigen-specific echographic contrast agent comprising:
at least two microspheres, different from each other and one located inside the other,
wherein each one of said microspheres is covered by a membrane, different for each microsphere and substantially constituted by an effective amount of at least one high affinity receptor specific for a certain antigen that is present in a tissue to be submitted to imaging or pharmaceutical treatment, and by an effective amount of at least one stabilizer, being the ponderal reciprocal ratio receptor:stabilizer comprised from 10:1 to 1:1; and
wherein each of said microspheres contains an inert gas, equal or different for each microsphere.

2. The contrast agent according to claim 1, wherein the contrast agents consists of two microspheres, said microspheres being substantially concentric.

3. The contrast agent according to claim 1, wherein said at least one high affinity receptor is selected from the group consisting of: monoclonal antibodies, non-monoclonal antibodies, fragments thereof, Fabs, Vascular Endothelial Growth Factor Receptors VEGFR, specific for endothelial cells, myocardial cells, lamina propria cells, interstitial cells, blood clot cells expressed in atherosclerotic plaques, specific receptors for immunoglobulines, complement fragments, peptidic or lipidic hormones, or neurotransmitters.

4. The contrast agent according to claim 1, wherein said at least one stabilizer is selected from the group consisting of: albumin, phospholipids, galactose, palmitic acid, cianoacrylate, or a mixture thereof.

5. The contrast agent according to claim 1, wherein said ponderal reciprocal ratio receptor:stabilizer is between 7.5:1 to 1.5:1.

6. The contrast agent according claim 1, wherein said membrane of said microspheres contains an amount of receptor up to about 91% by weight, with reference to a total weight of the membrane.

7. The contrast agent according to claim 1, wherein said inert gas is selected from the group consisting of: sulphur hexafluoride, perfluorohexane, perfluorocarbon, air, or nitrogen.

8. The contrast agent according to claim 1, wherein anyone of said at least two microspheres further contains or is coupled with an effective amount of at least one bio-active substance.

9. An echo-contrastographic pharmaceutical composition comprising:
an injectable aqueous suspension of an agent according to claim 1.

10. (canceled)

11. A method of specifically and selectively releasing a drug to a site, a tissue, an organ, or a district in a prolonged and gradual way, said method comprising:
administering to an organism an echo-contrastographic pharmaceutical composition according to claim 9; and
submitting said composition to specific and temporally differentiated cavitation, through application of ultrasonic beams different in potency intensity.

12. The method according to claim 11, wherein the at least one high affinity receptor in the echo-contrastographic pharmaceutical composition is selected from the group consisting of: monoclonal antibodies, non-monoclonal antibodies, fragments thereof, Fabs, Vascular Endothelial Growth Factor Receptors (VEGFR), specific for endothelial cells, myocardial cells, lamina propria cells, interstitial cells, blood clot cells expressed in atherosclerotic plaques, specific receptors for immunoglobulines, complement fragments, peptidic or lipidic hormones, or neurotransmitters.

13. The method according to claim 11, wherein the at least one stabilizer in the echo-contrastographic pharmaceutical composition is selected from the group consisting of: albumin, phospholipids, galactose, palmitic acid, cianoacrylate, or a mixture thereof.

14. The method according to claim 11, wherein the ponderal reciprocal ratio receptor:stabilizer in the echo-contrastographic pharmaceutical composition is between 7.5:1 to 1.5:1.

15. The method according claim 11, wherein the membrane of the microspheres in the echo-contrastographic pharmaceutical composition contains an amount of receptor up to about 91% by weight, with reference to a total weight of the membrane.

16. The method according to claim 11, wherein the inert gas in the echo-contrastographic pharmaceutical composition is selected from the group consisting of: sulphur hexafluoride, perfluorohexane, perfluorocarbon, air, or nitrogen.

17. The method according to claim 11, wherein anyone of the at least two microspheres in the echo-contrastographic pharmaceutical composition further contains or is coupled with an effective amount of at least one bio-active substance.
